(19) United States
(12) Patent Application Publication
(10) Pub. No.: US 2018/0042943 A1
(43) Pub. Date: Feb. 15, 2018

(54) METHODS FOR MODULATING BONE DENSITY

(71) Applicant: Intercept Pharmaceuticals, Inc., New York, NY (US)

(72) Inventors: David Shapiro, Santa Fe, CA (US); Luciano Adorini, Milano (IT)

(21) Appl. No.: 15/556,514

(22) PCT Filed: Mar. 8, 2016
(86) PCT No.: PCT/US16/21344

§ 371 Date: Sep. 7, 2017

Related U.S. Application Data
(60) Provisional application No. 62/130,144, filed on Mar. 9, 2015.

(51) Int. Cl. A61K 31/575; A61K 47/54

(52) U.S. Cl. CPC A61K 31/575; A61K 47/542

(57) ABSTRACT

The present invention relates to methods of treating, reducing the risk of, preventing, or alleviating a symptom of a disease or condition associated with changes in bone density, osteoporosis, or an osteopenic disease, or inducing osteogenesis or bone growth, or slowing, preventing, or reversing the reduction in bone density in a subject in need of treatment thereof, comprising administering a compound of the invention to the subject.

### BACKGROUND TO THE INVENTION

Bone is a dynamic tissue that is continually remodeled throughout life. Normal bone formation depends on the balance between bone addition and bone resorption, the former relying on the deposition of bone matrix by osteoblasts, and the latter being achieved by osteoclasts.

Bone resorption is initiated when an osteoclast attaches to the surface of bone, forms a tight “sealing zone”, and secretes necessary acids and proteases that initiate the resorption of mineralized tissue from the bone. After a period of several hours to days, the osteoclast detaches from the bone, leaving a “pit” on the bone surface. Under normal conditions, the pit is a target for osteoblasts, which deposit materials that ultimately become new bone. Bone loss can appear when the balance between bone addition and bone resorption is disturbed, for example, increased osteoclast activation, bone metastases, and bone erosions.

Various methods have been evaluated for increasing bone mass in humans, for instance in patients with osteoporosis. These treatments include administration of sodium fluoride, androgens, parathyroid hormone, calcitonin, and calcitonin in combination with high dietary phosphate. Except for treatment with sodium fluoride, the effects of these treatments are modest. Moreover, sodium fluoride treatment increases trabecular bone in some patients but has uncertain effects on total bone mass and bone strength, a high risk of osteomalacia, as well as other undesired side effects. None of these methods, however, have provided a clinically useful technique for increasing bone mass and often cause undesirable side effects.

Accordingly, there is a need for therapeutic agents having minimal side effects for the treatment of conditions involving the regulation of bone density. The present application addresses such a need.

### BRIEF DESCRIPTION OF THE DRAWINGS

FIG. 1 is a graph displaying the femoral bone density mean (SE) T-scores acquired by dual-emission x-ray absorptiometry scans at baseline and at twelve months.

FIG. 2 is bar graph displaying the femoral bone density LS mean (SE) change in T-score acquired by dual-emission x-ray absorptiometry scans at twelve months.

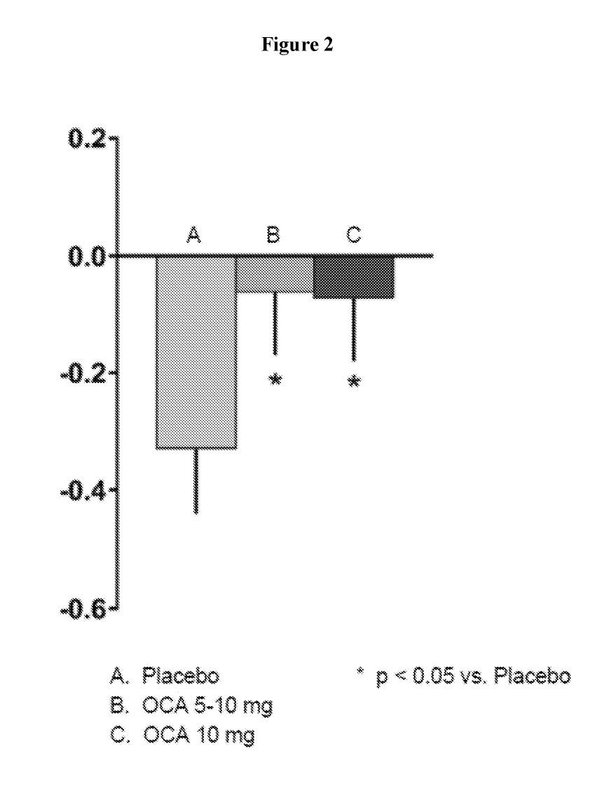

### SUMMARY OF THE INVENTION

The invention relates to a method of treating, reducing the risk of, preventing, or alleviating a symptom of a disease or condition associated with changes in bone density in a subject in need of treatment thereof, comprising administering to the subject a therapeutically effective amount of a compound of formula I:

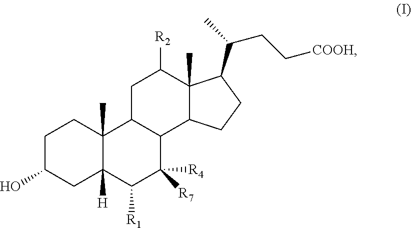

or a pharmaceutically acceptable salt or amino acid conjugate thereof, wherein R₁, R₂, R₄, and R₇ are as defined herein. In one aspect, the subject is suffering from a liver disease, such as primary biliary cirrhosis.

The invention also relates to a method of treating, reducing the risk of, preventing, or alleviating a symptom of osteoporosis or an osteopenic disease in a subject in need of treatment thereof, comprising administering to the subject a therapeutically effective amount of a compound of formula I, or a pharmaceutically acceptable salt or amino acid conjugate thereof, wherein R₁, R₂, R₄, and R₇ are as defined herein. In one aspect, the subject is suffering from a liver disease, such as primary biliary cirrhosis.

The invention also relates to a method of inducing osteogenesis or bone growth in a subject in need of treatment thereof, comprising administering to the subject a therapeutically effective amount of a compound of formula I, or a pharmaceutically acceptable salt or amino acid conjugate thereof, wherein R₁, R₂, R₄, and R₇ are as defined herein. In one aspect, the subject is suffering from a liver disease, such as primary biliary cirrhosis.

The invention also relates to a method of slowing, preventing, or reversing the reduction in bone density in a subject in need of treatment thereof, comprising administering to the subject a therapeutically effective amount of a compound of formula I, or a pharmaceutically acceptable salt or amino acid conjugate thereof, wherein R₁, R₂, R₄, and R₇ are as defined herein. In one aspect, the subject is suffering from a liver disease, such as primary biliary cirrhosis.

The invention also relates to use of a compound of formula I or a pharmaceutically acceptable salt or amino acid conjugate thereof, in the manufacture of a medicament for the treatment, reduction of the risk of, prevention, or alleviation of a symptom of a disease or condition associated with changes in bone density, osteoporosis, or an osteopenic disease, or for inducing osteogenesis or bone growth, or for slowing, preventing, or reversing the reduction in bone density, in a subject in need of treatment thereof, wherein R₁, R₂, R₄, and R₇ are as defined herein. In one aspect, the subject is suffering from a liver disease, such as primary biliary cirrhosis.

The invention also relates to a compound of formula I or a pharmaceutically acceptable salt or amino acid conjugate thereof, for the treatment, reduction of the risk of, prevention, or alleviation of a symptom of a disease or condition associated with changes in bone density, osteoporosis, or an osteopenic disease, or for inducing osteogenesis or bone growth, or for slowing, preventing, or reversing the reduction in bone density, in a subject, wherein R₁, R₂, R₄, and R₇ are as defined herein. In one aspect, the subject is suffering from a liver disease, such as primary biliary cirrhosis.

Unless otherwise defined, all technical and scientific terms used herein have the same meaning as commonly understood by one of ordinary skill in the art to which this invention belongs. In the case of conflict, the present specification, including definitions, will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In addition, the materials, methods, and examples are illustrative only and are not intended to be limiting.

Other features and advantages of the invention will be apparent from the following detailed description and claims.

### DETAILED DESCRIPTION OF THE INVENTION

The present application is directed to modulating bone density in a subject in need of treatment thereof, using a compound of formula I:

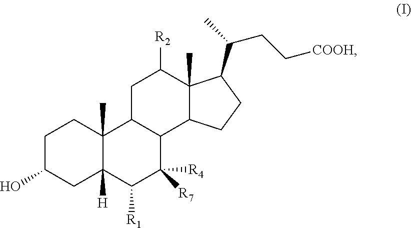

or a pharmaceutically acceptable salt or amino acid conjugate thereof, wherein:

R₁ is hydrogen or unsubstituted C₁-C₆ alkyl;

R₂ is hydrogen or α-hydroxyl;

R₄ is hydroxyl or hydrogen; and

R₇ is hydroxyl or hydrogen.

In one example, R₁ is unsubstituted C₁-C₆ alkyl. In a further example, R₁ is unsubstituted C₁-C₃ alkyl. In a further example, R₁ is methyl, ethyl, or propyl. In a further example, R₁ is ethyl.

In one example, R₂ is hydrogen. In another example, R₂ is α-hydroxyl.

In one example, R₄ is hydroxyl and R₇ is hydrogen. In another example, R₄ is hydrogen and R₇ is hydroxyl.

In a further example, R₁ is selected from methyl, ethyl and propyl, R₄ is hydroxyl, R₇ is hydrogen, and R₂ is hydrogen. In a further example, R₁ is ethyl.

In a further example, R₁ is selected from methyl, ethyl and propyl, R₄ is hydrogen, R₇ is hydroxyl, and R₂ is hydrogen. In a further example, R₁ is ethyl.

In a further example, R₁ is selected from methyl, ethyl and propyl, R₄ is hydroxyl, R₇ is hydrogen, and R₂ is α-hydroxyl. In a further example, R₁ is ethyl.

In a further example, R₁ is selected from methyl, ethyl and propyl, R₄ is hydrogen, R₇ is hydroxyl, and R₂ is α-hydroxyl. In a further example, R₁ is ethyl.

In one example, the amino acid conjugate is a glycine conjugate. In one example, the amino acid conjugate is a taurine conjugate.

In a further example, the compound is

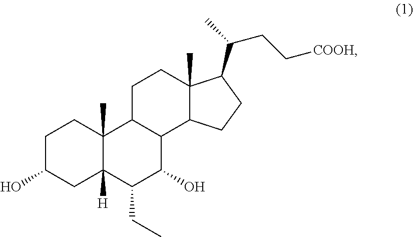 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt or amino acid conjugate thereof.

One of the solutions to the problem solved by the present invention is the identification of compounds as therapies for the treatment or prevention of conditions related to changes (e.g., reduction) in bone density, which can cause a number of diseases or disorders including, but not limited to, osteoporosis, osteopenia, Paget's disease of bone, osteomalacia, and osteopetrosis. Patients suffering from certain diseases or disorders may also develop conditions characterized by changes in bone density. In particular, conditions related to changes in bone density (e.g., osteoporosis or osteopenia) occur frequently in patients having an FXR mediated disease or condition. In treating patients with liver diseases, it is found that the compound of the present invention slows, prevents, or reverses the reduction in bone density, and/or induces osteogenesis or bone growth in patients.

In one aspect, the present invention relates to a method of treating, reducing the risk of, preventing, or alleviating a symptom of a disease or condition associated with changes in bone density in a subject in need of treatment thereof, comprising administering to the subject a therapeutically effective amount of a compound of formula I, or a pharmaceutically acceptable salt or amino acid conjugate thereof, wherein R₁, R₂, R₄, and R₇ are as defined herein.

In another aspect, the present invention relates to a method of treating, reducing the risk of, preventing, or alleviating a symptom of osteoporosis or an osteopenic disease in a subject in need of treatment thereof, comprising administering to the subject a therapeutically effective amount of a compound of formula I, or a pharmaceutically acceptable salt or amino acid conjugate thereof, wherein R₁, R₂, R₄, and R₇ are as defined herein.

In one example, the present invention relates to methods of treating a disease or condition associated with changes in bone density, osteoporosis, or an osteopenic disease. In another example, the present invention relates to methods of reducing the risk of a disease or condition associated with changes in bone density, osteoporosis, or an osteopenic disease. In another example, the present invention relates to methods of alleviating a symptom of a disease or condition associated with changes in bone density, osteoporosis, or an osteopenic disease.

In another aspect, the present invention relates to a method of inducing osteogenesis or bone growth in a subject in need of treatment thereof, comprising administering to the subject a therapeutically effective amount of a compound of formula I, or a pharmaceutically acceptable salt or amino acid conjugate thereof, wherein R₁, R₂, R₄, and R₇ are as defined herein.

In another aspect, the present invention relates to a method of slowing, preventing, or reversing the reduction in bone density in a subject in need of treatment thereof, comprising administering to the subject a therapeutically effective amount of a compound of formula I, or a pharmaceutically acceptable salt or amino acid conjugate thereof, wherein R₁, R₂, R₄, and R₇ are as defined herein.

In another aspect, the present invention relates to use of a compound of formula I or a pharmaceutically acceptable salt or amino acid conjugate thereof, in the manufacture of a medicament for the treatment, reduction of the risk of, prevention, or alleviation of a symptom of a disease or condition associated with changes in bone density, osteoporosis, or an osteopenic disease, or for inducing osteogenesis or bone growth, or for slowing, preventing, or reversing the reduction in bone density, in a subject in need of treatment thereof, wherein R₁, R₂, R₄, and R₇ are as defined herein.

In another aspect, the present invention relates to a compound of formula I or a pharmaceutically acceptable salt or amino acid conjugate thereof, for the treatment, reduction of the risk of, prevention, or alleviation of a symptom of a disease or condition associated with changes in bone density, osteoporosis, or an osteopenic disease, or for inducing osteogenesis or bone growth, or for slowing, preventing, or reversing the reduction in bone density, in a subject in need of treatment thereof, wherein R₁, R₂, R₄, and R₇ are as defined herein.

In one example, the subject has a decreased bone density as compared to a control subject. In a further example, the control subject is a young (e.g., 30 to 40 years old), healthy adult. In one example, the subject has a T-score that is 1.0 to 2.5 below that of a control subject. In another example, the subject has a T-score that is 2.5 below that of a control subject. In another further example, the control subject is a healthy adult having the same age as the subject. In a further example, the control subject is a healthy adult having the same age and ethnic background as the subject.

In one example, the methods of the present invention increase the T-score of the subject by at least 0.1, at least 0.2, at least 0.3, at least 0.4, at least 0.5, at least 0.6, at least 0.7, at least 0.8, at least 0.9, or at least 1.0, as compared to a control subject (e.g., a subject treated with placebo). In a further example, the methods of the present invention increase the T-score of the subject by at least 0.5, at least 0.6, at least 0.7, at least 0.8, at least 0.9, or at least 1.0, as compared to a control subject (e.g., a subject treated with placebo).

In one example, the methods of the present invention slow the reduction in bone density such that the T-score of the subject reduces by less than 0.5, less than 0.4, less than 0.3, less than 0.2, less than 0.1, less than 0.09, less than 0.08, less than 0.07, less than 0.06, less than 0.05, less than 0.04, less than 0.03, or less than 0.02 from the baseline (e.g., the T score measured before the treatment begins) during a time period of 6 months, 12 months, 18 months, or 24 months. In a further example, the methods of the present invention slow the reduction in bone density such that the T-score of the subject reduces by less than 0.3, less than 0.2, less than 0.1, less than 0.09, less than 0.08, less than 0.07, less than 0.06, less than 0.05, less than 0.04, less than 0.03, or less than 0.02 (e.g., the T score measured before the treatment begins) during a time period of 6 months, 12 months, 18 months, or 24 months. In a further example, the methods of the present invention slow the reduction in bone density such that the T-score of the subject reduces by less than 0.1, less than 0.09, less than 0.08, less than 0.07, less than 0.06, less than 0.05, less than 0.04, less than 0.03, or less than 0.02 (e.g., the T score measured before the treatment begins) during a treatment time period of 6 months, 12 months, 18 months, or 24 months. In one example, the time period is 12 months.

In one example, the subject is suffering from an FXR mediated disease or condition, such as those described herein. In one example, the subject is suffering from a liver disease or disorder, such as those described herein. In a further example, the subject is suffering from a cholestatic liver disease, such as those described herein. In a further example, the subject is suffering from PBC. In another further example, the subject is suffering from a non-cholestatic liver disease, such as those described herein.

In one example, the subject is suffering from an FXR mediated disease or condition. Examples of the FXR mediated diseases or conditions include, but are not limited to, liver diseases, renal diseases, pulmonary diseases, intestinal diseases, and cardiovascular diseases, in which FXR plays a role.

In one example, the subject is suffering from an FXR mediated liver disease. In one example, the subject is suffering from PBC.

In one example, the subject has an elevated level of liver enzymes, such as those described herein.

Examples of FXR mediated liver diseases include a cholestatic liver disease such as primary biliary cirrhosis (PBC), primary sclerosing cholangitis (PSC), biliary atresia, drug-induced cholestasis, hereditary cholestasis, intrahepatic cholestasis of pregnancy, and a cholestatic condition associated with a disease or condition such as primary liver and biliary cancer, metastatic cancer, sepsis, chronic total parenteral nutrition, cystic fibrosis, or granulomatous liver disease. In one example, a cholestatic condition is defined as having an abnormally elevated serum level of alkaline phosphatase, γ-glutamyl transpeptidase (GGT), and/or 5′ nucleotidase. In another example, a cholestatic condition is further defined as presenting with at least one clinical symptom. In a further example, the symptom is itching (pruritus). In a further example, a cholestatic condition is selected from the group consisting of PBC, PBS, drug-induced cholestasis, hereditary cholestasis, and intrahepatic cholestasis of pregnancy.

Examples of FXR mediated liver diseases also include portal hypertension, bile acid diarrhea, chronic liver disease, nonalcoholic fatty liver disease (NAFLD), nonalcoholic steatohepatitis (NASH), hepatitis C infection, alcoholic liver disease, liver damage due to progressive fibrosis, and liver fibrosis.

Examples of liver fibrosis include fibrosis associated with a disease such as hepatitis B; hepatitis C; parasitic liver diseases; post-transplant bacterial, viral and fungal infections; alcoholic liver disease (ALD); non-alcoholic fatty liver disease (NAFLD); non-alcoholic steatohepatitis (NASH), liver diseases induced by methotrexate, isoniazid, oxyphenistatin, methyldopa, chlorpromazine, tolbutamide, or amiodarone; autoimmune hepatitis; sarcoidosis; Wilson's disease; hemochromatosis; Gaucher's disease; types III, IV, VI, IX and X glycogen storage diseases; α1-antitrypsin deficiency; Zellweger syndrome; tyrosinemia; fructosemia; galactosemia; vascular derangement associated with Budd-Chiari syndrome, veno-occlusive disease, or portal vein thrombosis; or congenital hepatic fibrosis.

NAFLD is a medical condition that is characterized by the buildup of fat (called fatty infiltration) in the liver. NAFLD is one of the most common causes of chronic liver disease, and encompasses a spectrum of conditions associated with lipid deposition in hepatocytes. It ranges from steatosis (simple fatty liver), to nonalcoholic steatohepatitis (NASH), to advanced fibrosis and cirrhosis. The disease is mostly silent and is often discovered through incidentally elevated liver enzyme levels. NAFLD is strongly associated with obesity and insulin resistance and is currently considered by many as the hepatic component of the metabolic syndrome.

Nonalcoholic steatohepatitis (NASH) is a condition that causes inflammation and accumulation of fat and fibrous (scar) tissue in the liver. Liver enzyme levels in the blood may be more elevated than the mild elevations seen with nonalcoholic fatty liver (NAFL). Although similar conditions can occur in people who abuse alcohol, NASH occurs in those who drink little to no alcohol. NASH affects 2 to 5 percent of Americans, and is most frequently seen in people with one of more of the following conditions: obesity, diabetes, hyperlipidemia, insulin resistance, uses of certain medications, and exposure to toxins. NASH is an increasingly common cause of chronic liver disease worldwide and is associated with increased liver-related mortality and hepatocellular carcinoma, even in the absence of cirrhosis. NASH progresses to cirrhosis in 15-20% of affected individuals and is now one of the leading indications for liver transplantation in the United States. At present there are no approved therapies for NASH.

Examples of FXR mediated cardiovascular diseases include atherosclerosis, arteriosclerosis, hypercholesterolemia, and hyperlipidemia.

Examples of FXR mediated intestinal diseases include intestinal fibrosis associated with a disease such as Crohn's disease, ulcerative colitis, post-radiation colitis, or microscopic colitis.

Examples of FXR mediated renal diseases include renal fibrosis associated with a disease such as diabetic nephropathy, hypertensive nephrosclerosis, chronic glomerulonephritis, chronic transplant glomerulopathy, chronic interstitial nephritis, or polycystic kidney disease.

“Bone density”, “bone mineral density”, or BMD refers to the amount of mineral matter per square centimeter of bones. Bone density is an indirect indicator of various bone diseases or disorders, such as osteoporosis, and fracture risk. Many techniques have been developed to measure bone density. Generally, the measurement involves low radiation exposure and is painless and non-invasive. These techniques include conventional radiography, Dual-energy X-ray absorptiometry (DEXA), Single energy X-ray absorptiometry (SEXA), Dual photon absorptiometry (DPA), Single photon absorptiometry (SPA), Quantitative computed tomography (QCT), Digital X-ray radiogrammetry (DXR), and Qualitative ultrasound (QUS). Measurements are most commonly made over the lumbar spine and over the upper part of the hip.

Diseases, disorders, or conditions associated with changes in bone density include, but are not limited to, metabolic bone diseases, osteoporosis, osteopenia, Paget's disease of bone, osteomalacia, osteopetrosis, and hypophosphatasia.

The term “osteoporosis” refers to conditions in which decreased mineral or bone matrix and reduced bone mass occurs. In some cases, osteoporosis can be defined according to the World Health Organization (WHO) as a bone mineral density of 2.5 standard deviations or more below the mean peak bone mass (average of young, healthy adults) as measured by dual-energy X-ray absorptiometry. Diagnosis of osteoporosis can also be made using conventional radiography.

The term “osteopenic diseases” or “osteopenia” refers to conditions with decreased calcification and/or bone density, and is used to refer to all skeletal systems in which the condition is noted. In some cases, osteopenia can be defined according to the World Health Organization (WHO) as a bone mineral density of between 1.0 and 2.5 below the mean peak bone mass (average of young, healthy adults) as measured by dual-energy X-ray absorptiometry.

Paget's disease of bone or Paget disease of bone is a chronic disorder that can result in enlarged and misshapen bones. Paget's disease is caused by the excessive breakdown and formation of bone, followed by disorganized bone remodeling.

Osteomalacia refers to the softening of bones caused by defective bone mineralization due to deficiencies in bone formation, such as inadequate levels of phosphate and calcium available for bone growth, or because of over-active resorption of calcium from the bone as a result of hyperparathyroidism.

Osteopetrosis, also known as marble bone disease and Albers-Schonberg disease, refers to disorders whereby the bones harden, in contrast to more prevalent conditions like osteoporosis.

Dual-energy X-ray absorptiometry (DEXA) is considered the gold standard for the diagnosis of osteoporosis. Osteoporosis can be diagnosed when the bone mineral density is less than or equal to 2.5 standard deviations below that of a young (30 to 40-year-old), healthy adult reference population. The World Health Organization has established the following diagnostic guidelines.

| Category | T-score range | % young women |
|---|---|---|
| Normal | T-score ≧ −1.0 | 85% |
| Osteopenia | −2.5 < T-score < −1.0 | 14% |
| Osteoporosis | T-score ≦ −2.5 | 0.6% |
| Severe osteoporosis | T-score ≦ −2.5 with | |
| | fragility fracture | |

T-score is the relevant measure when screening for osteoporosis. It refers to the number of standard deviations of the bone mineral density (BMD) as measured when compared to the young normal reference mean (e.g., healthy thirty-year-old adults).

Z-score is the comparison to the age-matched normal and is usually used in cases of severe osteoporosis. This is the number of standard deviations a patient's BMD differs from the average BMD of their age, sex, and ethnicity. It is most useful when the T-score is less than 2 standard deviations below normal. In this setting, it is helpful to scrutinize for coexisting illnesses that may contribute to osteoporosis such as glucocorticoid therapy, hyperparathyroidism, or alcoholism.

The invention also comprehends an isotopically-labeled compound or a pharmaceutically acceptable salt or amino acid conjugate thereof, which has a structure that is identical to that of the compound of the present invention (e.g., a compound of formula I or Compound 1), but for the fact that one or more atoms are replaced by an atom having an atomic mass or mass number different from the atomic mass or mass number most commonly found in nature. Examples of isotopes that can be incorporated into the compound or a pharmaceutically acceptable salt or amino acid conjugate thereof, include isotopes of hydrogen, carbon, nitrogen, fluorine, such as ³H, ¹¹C, ¹⁴C and ¹⁸F.

The compound or a pharmaceutically acceptable salt or amino acid conjugate thereof that contain the aforementioned isotopes and/or other isotopes of other atoms is within the scope of the present invention. Isotopically-labeled compound or a pharmaceutically acceptable salt or amino acid conjugate thereof, for example, a compound into which a radioactive isotopes such as ³H and/or ¹⁴C are incorporated, is useful in drug and/or substrate tissue distribution assays. Tritiated, i.e., ³H, and carbon-14, i.e., ¹⁴C, isotopes are used for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be used in some circumstances. Isotopically labeled compound or a pharmaceutically acceptable salt or amino acid conjugate thereof can generally be prepared by carrying out the procedures disclosed in the Schemes and/or in the Examples of the invention, by substituting a readily available isotopically labeled reagent for a non-isotopically labeled reagent. In one example, obeticholic acid, or pharmaceutically acceptable salts or amino acid conjugates thereof are not isotopically labelled.

The present methods provides additional benefit of reducing the amount of bilirubin, and/or one or more liver enzymes in the subject.

In one example, the methods of the present application reduce the amount of bilirubin by at least 10%, 20%, 30%, 40%, 50%, 60%, 70%, 80%, or 90%, as compared to a control subject (e.g., a subject not administered with the composition of the present invention). In one example, the subject has an elevated level of bilirubin, as compared to a healthy subject (e.g., an individual without a disease or condition, such as those described herein). In one example, the methods of the present application reduce the level of bilirubin to a normal level (e.g., similar to the level of bilirubin in an individual without a disease or condition, such as those described herein). In a further example, the methods of the present application reduce the level of bilirubin below 10 mg/L, 9 mg/L, 8 mg/L, 7 mg/L, 6 mg/L, 5 mg/L, 4 mg/L, 3 mg/L, 2 mg/L, 1.5 mg/L, 1.2 mg/L, or 1 mg/L. In a further example, the methods of the present application reduce the level of bilirubin below 2 mg/L, 1.5 mg/L, 1.2 mg/L, or 1 mg/L.

In one example, the liver enzyme is selected from the group consisting of alkaline phosphatase (ALP, AP, or Alk Phos), alanine aminotransferase (ALT), aspartate aminotransferase (AST), gamma-glutamyl transpeptidase (GGT), lactate dehydrogenase (LDH), and 5′ nucleotidase. In one example, the methods of the present application reduce the amount of one or more liver enzymes by at least 10%, 20%, 30%, 40%, 50%, 60%, 70%, 80%, or 90%, as compared to a control subject (e.g., a subject not administered with the composition of the present invention). In one example, the subject has elevated levels of one or more liver enzymes, as compared to a healthy subject (e.g., an individual without a disease or condition, such as those described herein). In one example, the methods of the present application reduce the levels of one or more liver enzymes (e.g., ALP, ALT, AST, GGT, LDH, and 5′ nucleotidase) to normal levels (e.g., similar to the levels of liver enzymes in an individual without a disease or condition, such as those described herein).

In a further example, the methods of the present application reduce the level of ALP below 500 IU/L (international units per liter), 400 IU/L, 300 IU/L, 200 IU/L, 180 IU/L, 160 IU/L, or 150 IU/L. In a further example, the methods of the present application reduce the level of ALP to from about 40 IU/L to about 150 IU/L.

In a further example, the methods of the present application reduce the level of ALT below 200 IU/L (international units per liter), 150 IU/L, 100 IU/L, 80 IU/L, 60 IU/L, or 50 IU/L. In a further example, the methods of the present application reduce the level of ALT to from about 5 IU/L to about 50 IU/L.

In a further example, the methods of the present application reduce the level of AST below 200 IU/L (international units per liter), 150 IU/L, 100 IU/L, 80 IU/L, 60 IU/L, 50 IU/L, or 40 IU/L. In a further example, the methods of the present application reduce the level of AST to from about 10 IU/L to about 50 IU/L.

In a further example, the methods of the present application reduce the level of GGT below 200 IU/L (international units per liter), 150 IU/L, 100 IU/L, 90 IU/L, 80 IU/L, 70 IU/L, or 60 IU/L. In a further example, the methods of the present application reduce the level of GGT to from about 15 IU/L to about 50 IU/L or from about 5 IU/L to about 30 IU/L.

In a further example, the methods of the present application reduce the level of LDH below 500 IU/L (international units per liter), 400 IU/L, 300 IU/L, 200 IU/L, 180 IU/L, 160 IU/L, 150 IU/L, 140 IU/L, or 130 IU/L. In a further example, the methods of the present application reduce the level of LDH to from about 120 IU/L to about 220 IU/L.

In a further example, the methods of the present application reduce the level of 5′ nucleotidase below 50 IU/L (international units per liter), 40 IU/L, 30 IU/L, 20 IU/L, 18 IU/L, 17 IU/L, 16 IU/L, 15 IU/L, 14 IU/L, 13 IU/L, 12 IU/L, 11 IU/L, 10 IU/L, 9 IU/L, 8 IU/L, 7 IU/L, 6 IU/L, or 5 IU/L. In a further example, the methods of the present application reduce the level of 5′ nucleotidase to from about 2 IU/L to about 15 IU/L.

In one example, the subject is a mammal. In one example, the mammal is human.

In one example, the compound of the present invention is administered in a total daily amount from 1-25 mg, 2-20 mg, 3-15 mg, or 4-12 mg. In one example, the compound of the present invention is administered in an amount from about 5 mg (e.g., from 4.8 mg to 5.2 mg) to about 10 mg (e.g., from 9.8 mg to 10.2 mg). In one example, the compound of the present invention is administered in a total daily amount of about 5 mg (e.g., from 4.8 mg to 5.2 mg). In another example, the compound of the present invention is administered in an amount of about 10 mg (e.g., from 9.8 mg to 10.2 mg). In one example, the compound is administered for a period of from 1 month to 24 months, from 3 months to 20 months, from 5 months to 18 months, from 6 months to 12 months. In one example, the compound is administered for about 6 months. In one example, the compound is administered for about 12 months.

In one example, the compound of the present invention is administered at a first dose for a first time period, followed by administration of the compound at a second dose for a second time period. In one example, the compound or a pharmaceutically acceptable salt or amino acid conjugate thereof is administered in a total daily amount from 1-25 mg, 2-20 mg, 3-15 mg, or 4-12 mg for a first time period, followed by administration of the compound in an amount from 1-25 mg, 2-20 mg, 3-15 mg, or 4-12 mg. In one example, the first dose is different from the second dose. In a further example, the first dose is lower than the second dose. In another example, the first dose is higher than the second dose. In one example, the first dose is about 5 mg (e.g., from 4.8 mg to 5.2 mg), and the second dose is about 10 mg (e.g., from 9.8 mg to 10.2 mg). In one example, the first time period is from 1 month to 24 months, from 3 months to 20 months, from 5 months to 18 months, from 6 months to 12 months. In one example, the second time period is about 6 months. In one example, the second time period is from 1 month to 24 months, from 3 months to 20 months, from 5 months to 18 months, from 6 months to 12 months. In one example, the second time period is about 6 months.

In one example, the compound of the present application is administered orally, parenterally, or topically, together with a pharmaceutically acceptable carrier. In another example, the compound of the present application is administered orally.

In the methods of the present invention, the active substances may be administered in single daily doses, or in two, three, four or more identical or different divided doses per day, and they may be administered simultaneously or at different times during the day. Usually, the active substances will be administered simultaneously, more usually in a single combined dosage form.

The present application also relates to a pharmaceutical composition comprising the compound of the invention and one or more pharmaceutically acceptable carrier. A pharmaceutical composition of the present invention may be in any convenient form for oral administration, such as a tablet, capsule, powder, lozenge, pill, troche, elixir, lyophilized powder, solution, granule, suspension, emulsion, syrup or tincture. Slow-release or delayed-release forms may also be prepared, for example in the form of coated particles, multi-layer tablets, capsules within capsules, tablets within capsules, or microgranules.

Solid forms for oral administration may contain pharmaceutically acceptable binders, sweeteners, disintegrating agents, diluents, flavoring agents, coating agents, preservatives, lubricants and/or time delay agents. Suitable binders include gum acacia, gelatin, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavoring agents include peppermint oil, oil of wintergreen, cherry, orange or raspberry flavoring. Suitable coating agents include polymers or copolymers or acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulfite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further include dispersing agents and/or suspending agents. Suitable suspending agents include sodium carboxymethylcellulose, methylcellulose, hydroxypropyl methylcellulose, polyvinylpyrrolidone, sodium alginate or cetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further include one or more emulsifying agents. Suitable emulsifying agents include dispersing agents as exemplified above or natural gums such as gum acacia or gum tragacanth.

In some examples, the compound of the present invention is used either as an immediate release tablet or as a sustained release tablet. It is particularly effective when provided in a sustained release tablet. Sustained release tablets of various lipid lowering agents are commercially available. For prolonged action, the tablet is in a sustained release form.

In one example, the pharmaceutical compositions of the invention is a dosage form which comprises the compound of the present invention or a pharmaceutically acceptable salt or amino acid conjugate thereof in a total daily amount of from 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg. In one embodiment, the total amount is orally administered once a day.

The compounds disclosed herein can be prepared by conventional methods (e.g., those described in U.S. Publication No. 2009/0062526, U.S. Pat. No. 7,138,390, and WO 2006/122977), such as by a 6-step synthesis followed by one purification step to produce highly pure Compound 1 (obeticholic acid, or OCA) as shown in Scheme 1 below.

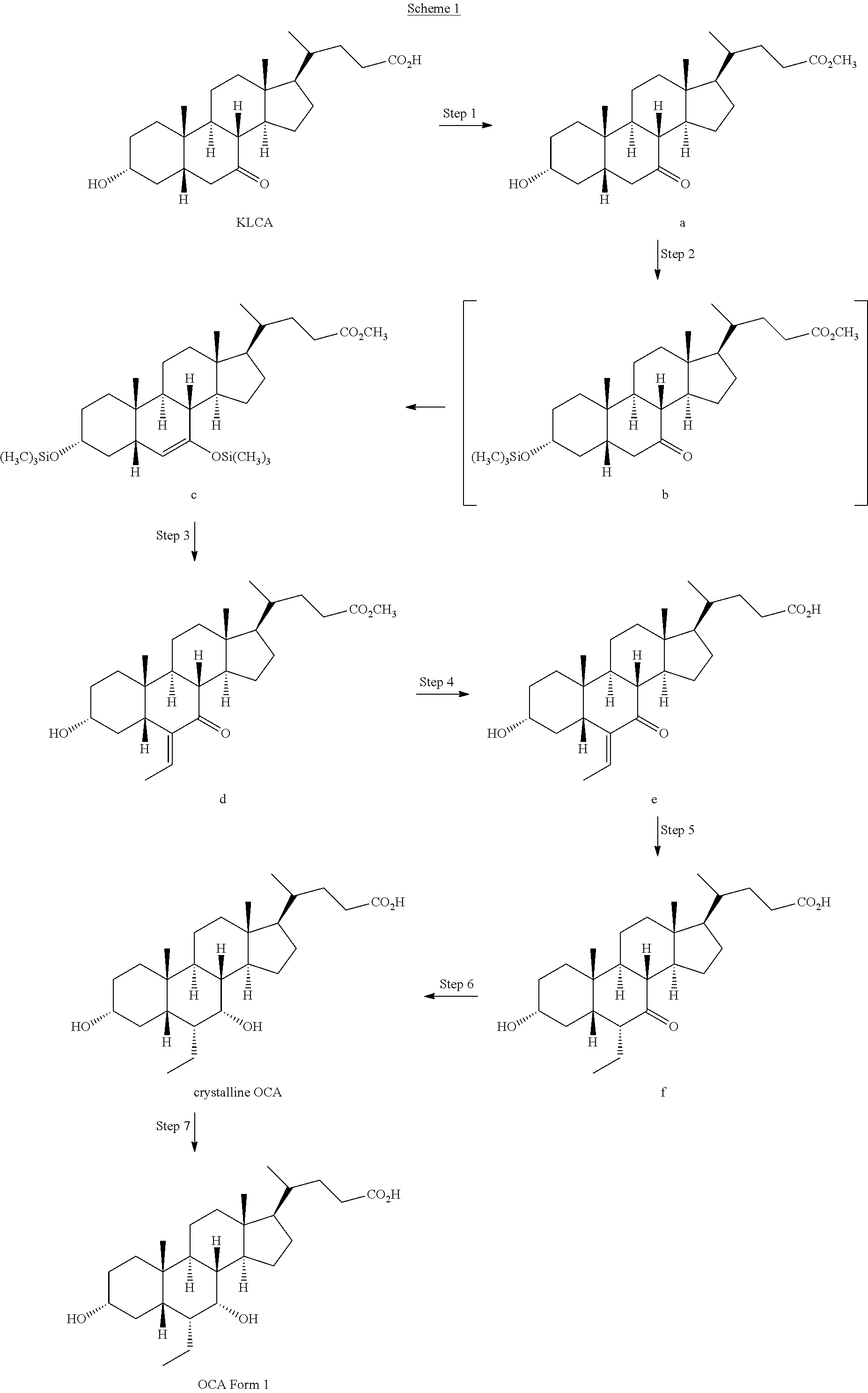

The process above was described in WO 2013/192097, the contents of which are incorporated herein by reference in their entirety. The process is a 6-step synthesis followed by one purification step. Step 1 is the esterification of the C-24 carboxylic acid of 7-keto lithocholic acid (KLCA) to produce the methyl ester compound a. Step 2 is silylenol ether formation from compound 1 to produce compound c. Step 3 is an aldol condensation reaction of the silylenol ether compound c and acetaldehyde to produce compound d. Step 4 is saponification of compound d to produce compound e. Step 5 is the hydrogenation of compound e to produce compound f. Step 6 is the selective reduction of the 7-keto group of compound f to produce crystalline Compound 1. Step 7 is the conversion of crystalline compound to Compound 1 (obeticholic acid Form 1, or OCA Form 1).

### Definitions

For convenience, certain terms used in the specification, examples and appended claims are collected here.

As used herein, the term “FXR agonist” refers to any compound which activates FXR. In one aspect, an FXR agonist achieves at least 50% activation of FXR relative to CDCA, the appropriate positive control in the assay methods described in WO 2000/037077. In another aspect, an FXR agonist achieves 100% activation of FXR in the scintillation proximity assay or the HTRF assay as described in WO2000/037077. Examples of FXR agonists include but are not limited to those described in U.S. Pat. No. 7,138,390; U.S. Pat. No. 7,932,244; 20120283234; 20120232116; 20120053163; 20110105475; 20100210660; 20100184809; 20100172870; 20100152166; 20100069367; 20100063018; 20100022498; 20090270460; 20090215748; 20090163474; 20090093524; 20080300235; 20080299118; 20080182832; 20080039435; 20070142340; 20060069070; 20050080064; 20040176426; 20030130296; 20030109467; 20030003520; 20020132223; and 20020120137.

As used herein, the term “obeticholic acid” or “OCA” refers to a compound having the chemical structure:

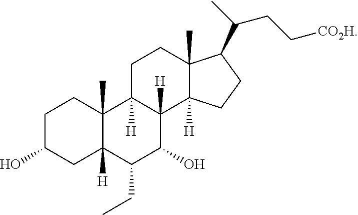

Obeticholic acid is also referred to as INT-747, 3α,7α-dihydroxy-6α-ethyl-5β-cholan-24-oic acid, 6α-ethyl-chenodeoxycholic acid, 6-ethyl-CDCA, or 6ECDCA, and can be prepared by the methods described in U.S. Publication No. 2009/0062526 A1, U.S. Pat. No. 7,138,390, and WO2006/122977. The CAS registry number for obeticholic acid is 459789-99-2.

As used herein, the term “crystalline obeticholic acid” refers to any crystalline form of a compound having the chemical structure:

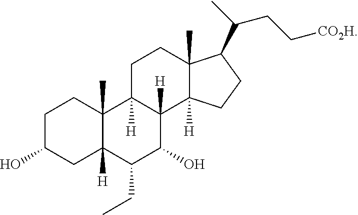

Crystalline obeticholic acid means that the compound is crystallized into a specific crystal packing arrangement in three spatial dimensions or the compound having external face planes. The crystalline form of obeticholic acid (or a pharmaceutically acceptable salt or an amino acid conjugate thereof) can crystallize into different crystal packing arrangements, all of which have the same elemental composition of obeticholic acid. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystals of obeticholic acid can be prepared by crystallization under different conditions, e.g., different solvents, temperatures, etc. Examples of crystalline forms of OCA are described in U.S. Pat. No. 9,238,673.

The term “compound(s) of the invention” or “compound(s) of the present invention” means a compound of formula I or Compound 1, or a pharmaceutically acceptable salt or amino acid conjugate thereof. Whenever the term is used in the context of the present invention it is to be understood that the reference is being made to the free acid, an isotopically-labeled compound, a crystalline compound, or a corresponding pharmaceutically acceptable salt or amino acid conjugate thereof, provided that such is possible and/or appropriate under the circumstances.

As used herein, the term “amino acid conjugates” refers to conjugates of the compound of the present invention (e.g., a compound of Formula I) with any suitable amino acid. For example, such a suitable amino acid conjugate of a compound of formula I may have the added advantage of enhanced integrity in bile or intestinal fluids. Suitable amino acids include but are not limited to glycine and taurine. Thus, the present invention encompasses the glycine and taurine conjugates of the compound of the present invention (e.g., Compound 1).

“Treating”, includes any effect, e.g., lessening, reducing, modulating, or eliminating, that results in the improvement of the condition, disease, disorder, etc. “Treating” or “treatment” of a disease state includes: inhibiting the disease state, i.e., arresting the development of the disease state or its clinical symptoms, or relieving the disease state, i.e., causing temporary or permanent regression of the disease state or its clinical symptoms.

“Preventing” the disease state includes causing the clinical symptoms of the disease state not to develop in a subject that may be exposed to or predisposed to the disease state, but does not yet experience or display symptoms of the disease state.

The term “inhibiting” or “inhibition,” as used herein, refers to any detectable positive effect on the development or progression of a disease or condition. Such a positive effect may include the delay or prevention of the onset of at least one symptom or sign of the disease or condition, alleviation or reversal of the symptom(s) or sign(s), and slowing or prevention of the further worsening of the symptom(s) or sign(s).

The term “effective amount” or “therapeutically effective amount” as used herein refers to an amount of the compound of the present invention (e.g., an FXR-activating ligand) that produces an acute or chronic therapeutic effect upon appropriate dose administration, alone or in combination. The effect includes the prevention, correction, inhibition, or reversal of the symptoms, signs and underlying pathology of a disease/condition (e.g., osteoporosis or osteopenia) and related complications to any detectable extent. An “effective amount” or “therapeutically effective amount” will vary depending on the compound, the disease and its severity, and the age, weight, etc., of the subject to be treated.

A therapeutically effective amount of the compound of the invention can be formulated with one or more pharmaceutically acceptable carriers for administration to a human or a non-human animal. Accordingly, the pharmaceutical composition of the invention can be administered, for example, via oral, parenteral, or topical routes, to provide an effective amount of the compound.

“Pharmacological effect” as used herein encompasses effects produced in the subject that achieve the intended purpose of a therapy. In one example, a pharmacological effect means that primary indications of the subject being treated are prevented, alleviated, or reduced. For example, a pharmacological effect would be one that results in the prevention, alleviation or reduction of primary indications in a treated subject. In another example, a pharmacological effect means that disorders or symptoms of the primary indications of the subject being treated are prevented, alleviated, or reduced. For example, a pharmacological effect would be one that results in the prevention, alleviation or reduction of the disorders or symptoms in a treated subject.

It is to be understood that the isomers arising from asymmetric carbon atoms (e.g., all enantiomers and diastereomers) are included within the scope of the invention, unless indicated otherwise. Such isomers can be obtained in substantially pure form by classical separation techniques and by stereochemically controlled synthesis.

A “pharmaceutical composition” is a formulation containing therapeutic agents such as the compound of the invention in a form suitable for administration to a subject. In one example, the pharmaceutical composition is in bulk or in unit dosage form. It can be advantageous to formulate compositions in dosage unit form for ease of administration and uniformity of dosage. Dosage unit form as used herein refers to physically discrete units suited as unitary dosages for the subject to be treated; each unit containing a predetermined quantity of active reagent calculated to produce the desired therapeutic effect in association with the required pharmaceutical carrier. The specification for the dosage unit forms of the invention are dictated by and directly dependent on the unique characteristics of the active agents and the particular therapeutic effect to be achieved, and the limitations inherent in the art of compounding such an active agent for the treatment of individuals.

The term “unit dosage form” refers to physically discrete units suitable as unitary dosages for humans and other mammals, each unit containing a predetermined quantity of active material calculated to produce the desired therapeutic effect, in association with a suitable pharmaceutical excipient as described herein.

The unit dosage form is any of a variety of forms, including, for example, a capsule, an IV bag, a tablet, a single pump on an aerosol inhaler, or a vial. The quantity of the compound of the invention or a pharmaceutically acceptable salt or amino acid conjugate thereof in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one example, the compound is mixed under sterile conditions with a pharmaceutically acceptable carrier, and with any preservatives, buffers, or propellants that are required.

The term “flash dose” refers to formulations that are rapidly dispersing dosage forms.

The term “immediate release” is defined as a release of a therapeutic agent (such as a the compound of the invention) from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a therapeutic agent from a dosage form over a prolonged period.

A “subject” includes mammals, e.g., humans, companion animals (e.g., dogs, cats, birds, and the like), farm animals (e.g., cows, sheep, pigs, horses, fowl, and the like), and laboratory animals (e.g., rats, mice, guinea pigs, birds, and the like). In one example, the subject is human. In one aspect, the subject is female. In one aspect, the subject is male.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier or excipient” means a carrier or excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

While it is possible to administer the compound of the invention directly without any formulation, the compound may be administered in the form of a pharmaceutical formulation comprising a pharmaceutically acceptable excipient. This formulation can be administered by a variety of routes including oral, buccal, rectal, intranasal, transdermal, subcutaneous, intravenous, intramuscular, and intranasal.

In one example, the compound of the invention can be administered transdermally. In order to administer transdermally, a transdermal delivery device (“patch”) is needed. Such transdermal patches may be used to provide continuous or discontinuous infusion of a compound of the present invention in controlled amounts. The construction and use of transdermal patches for the delivery of pharmaceutical agents is well known in the art. See, e.g., U.S. Pat. No. 5,023,252. Such patches may be constructed for continuous, pulsatile, or on demand delivery of pharmaceutical agents.

In one example, the pharmaceutical composition of the present invention is adapted for buccal and/or sublingual, or nasal administration. This example provides administration of the compound of the invention in a manner that avoids gastric complications, such as first pass metabolism by the gastric system and/or through the liver. This administration route may also reduce adsorption times, providing more rapid onset of therapeutic benefits.

The compound of the invention may be administered over a wide dosage range. In another example, the formulation comprises about 1 mg to about 30 mg of the compound. In another example, the formulation comprises about 4 mg to about 26 mg of the compound. In another example, the formulation comprises about 5 mg to about 25 mg of the compound. However, it will be understood that the amount of the compound actually administered will be determined by a physician, in the light of the relevant circumstances, including the condition to be treated, the chosen route of administration, the form of the compound administered, the lipid lowering agent(s) administered, the age, weight, and response of the individual patient, and the severity of the patient's symptoms. Therefore, the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

“Fibrosis” refers to a condition involving the development of excessive fibrous connective tissue, e.g., scar tissue, in a tissue or organ. Such generation of scar tissue may occur in response to infection, inflammation, or injury of the organ due to a disease, trauma, chemical toxicity, and so on. Fibrosis may develop in a variety of different tissues and organs, including the liver, kidney, intestine, lung, heart, etc.

As used herein, a “cholestatic condition” refers to any disease or condition in which bile excretion from the liver is impaired or blocked, which can occur either in the liver or in the bile ducts. Intrahepatic cholestasis and extrahepatic cholestasis are the two types of cholestatic conditions. Intrahepatic cholestasis (which occurs inside the liver) is most commonly seen in primary biliary cirrhosis, primary sclerosing cholangitis, sepsis (generalized infection), acute alcoholic hepatitis, drug toxicity, total parenteral nutrition (being fed intravenously), malignancy, cystic fibrosis, and pregnancy. Extrahepatic cholestasis (which occurs outside the liver) can be caused by bile duct tumors, strictures, cysts, diverticula, stone formation in the common bile duct, pancreatitis, pancreatic tumor or pseudocyst, and compression due to a mass or tumor in a nearby organ.

Clinical symptoms and signs of a cholestatic condition include: itching (pruritus), fatigue, jaundiced skin or eyes, inability to digest certain foods, nausea, vomiting, pale stools, dark urine, and right upper quadrant abdominal pain. A patient with a cholestatic condition can be diagnosed and followed clinically based on a set of standard clinical laboratory tests, including measurement of levels of alkaline phosphatase, γ-glutamyl transpeptidase (GGT), 5′ nucleotidase, bilirubin, bile acids, and cholesterol in a patient's blood serum. Generally, a patient is diagnosed as having a cholestatic condition if serum levels of all three of the diagnostic markers alkaline phosphatase, GGT, and 5′ nucleotidase, are considered abnormally elevated. The normal serum level of these markers may vary to some degree from laboratory to laboratory and from procedure to procedure, depending on the testing protocol. Thus, a physician will be able to determine, based on the specific laboratory and test procedure, what an abnormally elevated blood level is for each of the markers. For example, a patient suffering from a cholestatic condition generally has greater than about 125 IU/L alkaline phosphatase, greater than about 65 IU/L GGT, and greater than about 17 NIL 5″nucleotidase in the blood. Because of the variability in the level of serum markers, a cholestatic condition may be diagnosed on the basis of abnormal levels of these three markers in addition to at least one of the symptoms mentioned above, such as itching (pruritus).

The term “primary biliary cirrhosis”, often abbreviated PBC, is an autoimmune disease of the liver marked by the slow progressive destruction of the small bile ducts of the liver, with the intralobular ducts (Canals of Hering) affected early in the disease. When these ducts are damaged, bile builds up in the liver (cholestasis) and over time damages the tissue. This can lead to scarring, fibrosis and cirrhosis. Primary biliary cirrhosis is characterized by interlobular bile duct destruction. Histopathologic findings of primary biliary cirrhosis include: inflammation of the bile ducts, characterized by intraepithelial lymphocytes, and periductal epithelioid granulomata. There are 4 stage of PBC.

Stage 1—Portal Stage: Normal sized triads; portal inflammation, subtle bile duct damage. Granulomas are often detected in this stage.

Stage 2—Periportal Stage: Enlarged triads; periportal fibrosis and/or inflammation. Typically this stage is characterized by the finding of a proliferation of small bile ducts.

Stage 3—Septal Stage: Active and/or passive fibrous septa.

Stage 4—Biliary Cirrhosis: Nodules present; garland

The term “primary sclerosing cholangitis” (PSC) is a disease of the bile ducts that causes inflammation and subsequent obstruction of bile ducts both at a intrahepatic (inside the liver) and extrahepatic (outside the liver) level. The inflammation impedes the flow of bile to the gut, which can ultimately lead to cirrhosis of the liver, liver failure and liver cancer.

The term “Nonalcoholic steatohepatitis” (NASH) is liver inflammation caused by a buildup of fat in the liver. In some people, the buildup of fat causes inflammation of the liver. Because of the inflammation, the liver doesn't work as well as it should. NASH can get worse and cause scarring of the liver, which leads to cirrhosis. NASH is similar to the kind of liver disease that is caused by long-term, heavy drinking, but NASH occurs in people who do not abuse alcohol.

The term “organ” refers to a differentiated structure (as in a heart, lung, kidney, liver, etc.) consisting of cells and tissues and performing some specific function in an organism. This term also encompasses bodily parts performing a function or cooperating in an activity (e.g., an eye and related structures that make up the visual organs). The term “organ” further encompasses any partial structure of differentiated cells and tissues that is potentially capable of developing into a complete structure (e.g., a lobe or a section of a liver).

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of examples and that the description and examples provided herein are for purposes of illustration and not limitation of the claims that follow.

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the present specification will control. All percentages and ratios used herein, unless otherwise indicated, are by weight.

### EXAMPLES

### Example 1

### General Protocols

### Patient Screening

Patients are screened during a ≦1 to 8 week period prior to treatment to allow for the collection of repeat serum chemistry samples (at least 2 weeks apart), if necessary, to confirm pretreatment ALP and total bilirubin values. Eligible patients are randomized to three groups: (a), (b), or (c) as described below.

All of the following must be met for being eligible for treatment.
1. Definite or probable PBC diagnosis (consistent with AASLD and EASL Practice Guidelines; [Lindor 2009; EASL 2009]), as demonstrated by the presence of ≧2 of the following 3 diagnostic factors:
   History of elevated ALP levels for at least 6 months
   Positive AMA titer or PBC specific antibodies
   Liver biopsy consistent with PBC
2. At least 1 of the following qualifying biochemistry values:
   ALP≧1.67×ULN
   Total bilirubin>ULN but<2×ULN
3. Age≧18 years
4. Taking UDCA for at least 12 months (stable dose for ≧3 months) prior to Day 0, or unable to tolerate UDCA (no UDCA for ≧3 months) prior to Day 0
5. Contraception: Female patients of childbearing potential must use ≧1 effective (≦1% failure rate) method of contraception during the treatment and for 30 days after the EOT visit.
6. Must provide written informed consent and agree to comply with the treatment protocol.

Patients are excluded from the treatment if they meet any of the following:
1. History or presence of other concomitant liver diseases including:
   Hepatitis C virus (HCV) infection
   Patients with active hepatitis B (HBV) infection are excluded, however, patients who have seroconverted (Hbs Ag and Hbe Ag negative) may be included after consultation with the medical monitor
   Primary sclerosing cholangitis (PSC)
   Alcoholic liver disease
   Definite autoimmune liver disease or overlap hepatitis
   Nonalcoholic steatohepatitis (NASH)
   Gilbert's Syndrome (exclusion due to interpretability of bilirubin levels)
2. Presence of clinical complications of PBC or clinically significant hepatic decompensation, including:
   History of liver transplantation, current placement on a liver transplant list or current MELD score≧15
   Portal hypertension with complications, including: known gastric or large esophageal varices, poorly controlled or diuretic resistant ascites, history of variceal bleeds or related therapeutic or prophylactic interventions (e.g., beta blockers, insertion of variceal bands or transjugular intrahepatic portosystemic shunt [TIPS]), or hepatic encephalopathy
   Cirrhosis with complications, including history or presence of: spontaneous bacterial peritonitis, hepatocellular carcinoma, bilirubin>2×ULN
   Hepatorenal syndrome (type I or II) or Screening serum creatinine>2 mg/dL (178 μmol/L)
3. Severe pruritus or those requiring systemic treatment for pruritus (e.g., with bile acid sequestrants [BAS] or rifampicin) within 2 months of Day 0
4. Administration of the following medications is prohibited as specified below:
   Prohibited 6 months prior to Day 0 and throughout the treatment (i.e., to last dose and/or EOT): azathioprine, colchicine, cyclosporine, methotrexate, mycophenolate mofetil, pentoxifylline; fenofibrate or other fibrates; budesonide and other systemic corticosteroids; potentially hepatotoxic drugs (including α-methyl-dopa, sodium valproic acid, isoniazide, or nitrofurantoin)
   Prohibited 12 months prior to Day 0 and throughout the treatment (i.e., to last dose and/or EOT): antibodies or immunotherapy directed against interleukins or other cytokines or chemokines
5. Previously participated in a clinical treatment of OCA
6. History or presence of clinically concerning cardiac arrhythmias likely to affect survival during the treatment, or prolongation of Screening (pretreatment) QT or QTc interval of >500 milliseconds (msec)
7. If female: known pregnancy, or has a positive urine pregnancy test (confirmed by a positive serum pregnancy test), or lactating
8. Known history of human immunodeficiency virus (HIV) infection
9. Presence of any other disease or condition that is interfering with the absorption, distribution, metabolism, or excretion of drugs including bile salt metabolism in the intestine. Patients with inflammatory bowel disease or who have undergone gastric bypass procedures will be excluded (gastric lap band is acceptable)
10. Medical conditions that may cause nonhepatic increases in ALP (e.g., Paget's disease) or which may diminish life expectancy to <2 years, including known cancers (except carcinomas in situ or other stable, relatively benign conditions such as chronic lymphatic leukemia)
11. Other clinically significant medical conditions that are not well controlled or for which medication needs are anticipated to change during the treatment
12. Anticipated changes to current concomitant medications during the course of the treatment
13. History of alcohol abuse, defined as consumption of more than 210 mL of alcohol per week (i.e., the equivalent of 14 4-ounce (125 mL) glasses of wine or 14 12-ounce cans/bottles of beer), or other substance abuse within 1 year prior to Day 0
14. Participation in another investigational drug, biologic, or medical device trial within 30 days prior to Screening
15. History of noncompliance with medical regimens, or considered to be potentially unreliable
16. Blood or plasma donation within 30 days prior to Day 0
17. Mental instability or incompetence, such that the validity of informed consent or compliance with the trial is uncertain

### Patient Stratification

Prior to randomization, patients are stratified by the presence or absence of the following 2 factors and randomized, in equal proportions, to each of the treatment groups:
1. Pretreatment ALP>3×ULN and/or AST>2×ULN and/or bilirubin>ULN
2. Intolerance to UDCA treatment

### Patient Randomization

PBC patients are randomized to three groups: (a) placebo (PCB), (b) 10 mg OCA, or (c) 5 mg (month 0-6) titrating to 10 mg (month 7-12) OCA. Study medication is administered orally, once daily for 12 months. For patients under pretreatment ursodeoxycholic acid (UDCA) treatment, the pre-treatment dose of UDCA is continued throughout their treatment.

‘Baseline’ (BL) within this protocol, unless otherwise specified, is intended to mean, ‘prestudy’ or ‘pretreatment’ (of study medication). It refers to values obtained during the Screening or Day 0 visits, prior to the patient's first dose of study medication. The statistical or calculated definition(s) of ‘baseline’ to be used in the analyses of the data may be different and will be further defined in the statistical analysis plan (SAP) for this treatment.

### Bone Density Assessment

Bone density is measured by DEXA scans. DEXA scans of the lumbar spine and femoral neck are conducted at Day 0 and Month 12. Additional measurement may be conducted after Month 12 (e.g., annually after Month 12) as necessary and appropriate. The timing of the DEXA scans is not critical, and may be conducted±2 weeks. Patients that have had a recent DEXA scan within 6 months prior to Day 0 and for which a report of the results is available for use in the treatment, do not need to repeat the baseline DEXA scan.

### Example 2

Subjects with PBC±UDCA (if taking UDCA, patients were maintained on a stable dose) with ALP≧1.67×ULN or bilirubin<2×ULN were randomized to placebo (PBO), OCA 5 or 10 mg for 12 months. Subjects on 5 mg were titrated to 10 mg after 6 months (OCA Titration) based on clinical response and tolerability. Dual-emission X-ray absorptiometry (DEXA) scan was used to assess BMD in a subset of subjects prior to and following 12 months of OCA or placebo treatment. Results of the femoral neck and lumbar spine (using T-score, Z-score, and BMD) were summarized in the Tables 1-6 below. Changes from baseline at Month 12 were analyzed using an ANCOVA model with baseline values as a covariate. Osteopenia and osteoporosis were based on WHO thresholds: T score −1.0 to ≦−2.5 and respectively.

Of the 216 subjects enrolled in the trial, 122 had DEXA scans at baseline and Month 12 (85% Female; 22%≧65 years of age; 52% postmenopausal). Baseline ALP was 318±102 U/L and 91% of subjects took concomitant UDCA. At baseline the prevalence of osteopenia and osteoporosis was 7% and 54%, respectively. Placebo subjects had a statistically significant decrease in Femoral T-scores from baseline to 12 months, as compared to OCA 10 mg (p=0.03) and to the Titration OCA (p=0.02). No significant difference between treatment groups were seen in Lumbar BMD. Results were generally consistent but did not attain statistical significance when assessed based on menopausal status.

TABLE 1
Femoral Neck Scan: T-Score

| | | Placebo (N = 73) | | Titration OCA (N = 70) | | 10 mg OCA (N = 73) | |
|---|---|---|---|---|---|---|---|
| | | | Change | | Change | | Change |
| | | | From | | From | | From |
| Parameter | Time Point | Result | Baseline | Result | Baseline | Result | Baseline |
| | Baseline^{[1]} | | | | | | |
| | n | 46 | | 48 | | 44 | |
| | Mean (SD) | −1.15 | | −1.29 | | −0.89 | |
| | | (1.172) | | (0.954) | | (1.041) | |
| | SEM | 0.173 | | 0.138 | | 0.157 | |
| | Median | −1.25 | | −1.30 | | −1.05 | |
| | Min, Max | −4.2, 2.2 | | −3.6, 1.8 | | −2.5, 3.6 | |
| | DB Month 12 | | | | | | |
| | n | 42 | 36 | 42 | 40 | 38 | 36 |
| | Mean (SD) | −1.48 | −0.32 | −1.28 | −0.03 | −1.06 | −0.11 |
| | | (1.036) | (0.836) | (0.946) | (0.316) | (0.820) | (0.235) |
| | SEM | 0.160 | 0.139 | 0.146 | 0.050 | 0.133 | 0.039 |
| | Median | −1.55 | −0.10 | −1.45 | −0.05 | −1.05 | −0.10 |
| | Min, Max | −4.3, 2.4 | −3.9, 0.4 | −2.8, 2.2 | −0.7, 0.8 | −3.0, 0.9 | −0.6, 0.5 |
| | LS Mean | | −0.33 | | −0.06 | | −0.07 |
| | (StdErr) | | (0.11) | | (0.11) | | (0.11) |
| | 95% CI | | (−0.55, −0.10) | | (−0.28, 0.16) | | (−0.29, 0.15) |
| | OCA - | | | | 0.27 | | 0.26 |
| | Placebo | | | | (0.11) | | (0.12) |
| | (StdErr) | | | | | | |
| | 95% CI of | | | | (0.05, 0.50) | | (0.03, 0.49) |
| | Mean | | | | | | |
| | Difference | | | | | | |
| | p-value^{[2]} | | | | 0.0184 | | 0.0302 |

^{[1]}Baseline is defined as the Day 0 value prior to treatment.
^{[2]}P-value for comparing active treatments to placebo is obtained using an ANCOVA model with baseline value as a covariate and fixed effects for treatment and randomization strata factor.

TABLE 2
Femoral Neck Scan: Z-Score

| | | | | Titration OCA | | | |
|---|---|---|---|---|---|---|---|
| | | Placebo (N = 73) | | (N = 70) | | 10 mg OCA (N = 73) | |
| | | | Change | | Change | | Change |
| | | | From | | From | | From |
| Parameter | Time Point | Result | Baseline | Result | Baseline | Result | Baseline |
| | Baseline^{[1]} | | | | | | |
| | n | 44 | | 47 | | 43 | |
| | Mean (SD) | −0.32 | | −0.27 | | 0.10 | |
| | | (0.893) | | (0.927) | | (0.919) | |
| | SEM | 0.135 | | 0.135 | | 0.140 | |
| | Median | −0.30 | | −0.40 | | 0.00 | |
| | Min, Max | −2.0, 2.9 | | −2.0, 3.2 | | −1.5, 3.8 | |
| | DB Month 12 | | | | | | |
| | n | 40 | 34 | 42 | 39 | 37 | 34 |
| | Mean (SD) | −0.37 | −0.10 | −0.23 | 0.01 | −0.01 | −0.06 |
| | | (0.982) | (0.344) | (0.909) | (0.299) | (0.752) | (0.250) |
| | SEM | 0.155 | 0.059 | 0.140 | 0.048 | 0.124 | 0.043 |
| | Median | −0.40 | −0.05 | −0.45 | 0.00 | −0.10 | 0.00 |
| | Min, Max | −2.1, 3.1 | −0.9, 0.7 | −1.7, 3.6 | −0.7, 0.6 | −1.4, 2.2 | −0.6, 0.5 |
| | LS Mean | | −0.11 | | −0.01 | | −0.07 |
| | (StdErr) | | (0.07) | | (0.07) | | (0.07) |
| | 95% CI | | (−0.26, 0.03) | | (−0.15, 0.13) | | (−0.21, 0.07) |
| | OCA - | | | | 0.10 | | 0.05 |
| | Placebo | | | | (0.07) | | (0.08) |
| | (StdErr) | | | | | | |
| | 95% CI of | | | | (−0.04, 0.25) | | (−0.10, 0.20) |
| | Mean | | | | | | |
| | Difference | | | | | | |
| | p-value^{[2]} | | | | 0.1479 | | 0.5440 |

^{[1]}Baseline is defined as the Day 0 value prior to treatment.
^{[2]}P-value for comparing active treatments to placebo is obtained using an ANCOVA model with baseline value as a covariate and fixed effects for treatment and randomization strata factor.

TABLE 3
Femoral Neck Scan: Bone Mineral Density (g/cm²)

| | | Placebo (N = 73) | | Titration OCA (N = 70) | | 10 mg OCA (N = 73) | |
|---|---|---|---|---|---|---|---|
| | | | Change | | Change | | Change |
| | | | From | | From | | From |
| Parameter | Time Point | Result | Baseline | Result | Baseline | Result | Baseline |
| | Baseline^{[1]} | | | | | | |
| | n | 45 | | 48 | | 41 | |
| | Mean (SD) | 0.787 | | 0.804 | | 0.865 | |
| | | (0.131) | | (0.124) | | (0.163) | |
| | SEM | 0.020 | | 0.018 | | 0.025 | |
| | Median | 0.799 | | 0.816 | | 0.851 | |
| | Min, Max | 0.476, 1.091 | | 0.525, 1.195 | | 0.610, 1.408 | |
| | DB Month 12 | | | | | | |
| | n | 40 | 33 | 41 | 39 | 36 | 34 |
| | Mean (SD) | 0.763 | −0.011 | 0.809 | −0.003 | 0.813 | −0.034 |
| | | (0.134) | (0.049) | (0.133) | (0.056) | (0.151) | (0.054) |
| | SEM | 0.021 | 0.009 | 0.021 | 0.009 | 0.025 | 0.009 |
| | Median | 0.755 | −0.010 | 0.822 | −0.004 | 0.779 | −0.018 |
| | Min, Max | 0.465, 1.111 | −0.188, 0.130 | 0.534, 1.242 | −0.199, 0.172 | 0.591, 1.193 | −0.193, 0.032 |
| | LS Mean | | −0.02 | | −0.01 | | −0.04 |
| | (StdErr) | | (0.01) | | (0.01) | | (0.01) |
| | 95% CI | | (−0.04, 0.01) | | (−0.03, 0.02) | | (−0.06, −0.01) |
| | OCA - | | | | 0.01 | | −0.02 |
| | Placebo | | | | (0.01) | | (0.01) |
| | (StdErr) | | | | | | |
| | 95% CI of | | | | (−0.01, 0.04) | | (−0.04, 0.01) |
| | Mean | | | | | | |
| | Difference | | | | | | |
| | p-value^{[2]} | | | | 0.3812 | | 0.2112 |

^{[1]}Baseline is defined as the Day 0 value prior to treatment.
^{[2]}P-value for comparing active treatments to placebo is obtained using an ANCOVA model with baseline value as a covariate and fixed effects for treatment and randomization strata factor.

TABLE 4
Lumbar Spine Scan: T-Score

| | | | | Titration OCA | | | |
|---|---|---|---|---|---|---|---|
| | | Placebo (N = 73) | | (N = 70) | | 10 mg OCA (N = 73) | |
| | | | Change | | Change | | Change |
| | | | From | | From | | From |
| Parameter | Time Point | Result | Baseline | Result | Baseline | Result | Baseline |
| | Baseline^{[1]} | | | | | | |
| | n | 47 | | 49 | | 44 | |
| | Mean (SD) | −1.16 | | −1.10 | | −0.82 | |
| | | (1.469) | | (1.506) | | (1.299) | |
| | SEM | 0.214 | | 0.215 | | 0.196 | |
| | Median | −1.40 | | −1.40 | | −1.00 | |
| | Min, Max | −4.8, 2.9 | | −3.1, 6.1 | | −3.1, 2.5 | |
| | DB Month 12 | | | | | | |
| | n | 44 | 43 | 42 | 42 | 38 | 38 |
| | Mean (SD) | −1.42 | −0.30 | −1.10 | −0.06 | −1.02 | −0.17 |
| | | (1.383) | (0.951) | (1.635) | (0.284) | (1.297) | (0.428) |
| | SEM | 0.209 | 0.145 | 0.252 | 0.044 | 0.210 | 0.069 |
| | Median | −1.50 | −0.20 | −1.35 | 0.00 | −1.20 | −0.10 |
| | Min, Max | −5.0, 1.3 | −5.8, 0.6 | −3.0, 6.3 | −0.7, 0.8 | −3.5, 2.3 | −1.9, 0.5 |
| | LS Mean | | −0.26 | | −0.01 | | −0.09 |
| | (StdErr) | | (0.14) | | (0.14) | | (0.14) |
| | 95% CI | | (−0.53, 0.02) | | (−0.28, 0.27) | | (−0.37, 0.18) |
| | OCA - | | | | 0.25 | | 0.16 |
| | Placebo | | | | (0.14) | | (0.14) |
| | (StdErr) | | | | | | |
| | 95% CI of | | | | (−0.02, 0.52) | | (−0.12, 0.44) |
| | Mean | | | | | | |
| | Difference | | | | | | |
| | p-value^{[2]} | | | | 0.0682 | | 0.2523 |

^{[1]}Baseline is defined as the Day 0 value prior to treatment.
^{[2]}P-value for comparing active treatments to placebo is obtained using an ANCOVA model with baseline value as a covariate and fixed effects for treatment and randomization strata factor.

TABLE 5
Lumbar Spine Scan: Z-Score

| | | | | Titration OCA | | | |
|---|---|---|---|---|---|---|---|
| | | Placebo (N = 73) | | (N = 70) | | 10 mg OCA (N = 73) | |
| | | | Change | | Change | | Change |
| | | | From | | From | | From |
| Parameter | Time Point | Result | Baseline | Result | Baseline | Result | Baseline |
| | Baseline^{[1]} | | | | | | |
| | n | 45 | | 48 | | 43 | |
| | Mean (SD) | −0.32 | | −0.06 | | 0.11 | |
| | | (1.164) | | (1.735) | | (1.260) | |
| | SEM | 0.174 | | 0.250 | | 0.192 | |
| | Median | −0.40 | | −0.35 | | 0.00 | |
| | Min, Max | −3.0, 2.7 | | −2.0, 7.7 | | −2.6, 3.0 | |
| | DB Month 12 | | | | | | |
| | n | 42 | 38 | 42 | 41 | 37 | 36 |
| | Mean (SD) | −0.26 | 0.02 | 0.02 | 0.00 | 0.01 | −0.03 |
| | | (1.223) | (0.447) | (1.800) | (0.282) | (1.166) | (0.368) |
| | SEM | 0.189 | 0.073 | 0.278 | 0.044 | 0.192 | 0.061 |
| | Median | −0.40 | 0.00 | −0.20 | 0.00 | 0.00 | 0.00 |
| | Min, Max | −3.0, 2.5 | −0.9, 2.2 | −2.0, 7.8 | −0.7, 0.9 | −2.3, 3.3 | −1.2, 1.3 |
| | LS Mean | | 0.02 | | 0.00 | | −0.03 |
| | (StdErr) | | (0.09) | | (0.08) | | (0.09) |
| | 95% CI | | (−0.16, | | (−0.16, 0.17) | | (−0.20, 0.14) |
| | | | 0.19) | | | | |
| | OCA - | | | | −0.01 | | −0.05 |
| | Placebo | | | | (0.09) | | (0.09) |
| | (StdErr) | | | | | | |
| | 95% CI of | | | | (−0.18, 0.15) | | (−0.22, 0.13) |
| | Mean | | | | | | |
| | Difference | | | | | | |
| | p-value^{[2]} | | | | 0.8681 | | 0.6025 |

^{[1]}Baseline is defined as the Day 0 value prior to treatment.
^{[2]}P-value for comparing active treatments to placebo is obtained using an ANCOVA model with baseline value as a covariate and fixed effects for treatment and randomization strata factor.

TABLE 6
Lumbar Spine Scan: Bone Mineral Density (g/cm²)

| | | Placebo (N = 73) | | Titration OCA (N = 70) | | 10 mg OCA (N = 73) | |
|---|---|---|---|---|---|---|---|
| | | | Change | | | | Change |
| | | | From | | Change From | | From |
| Parameter | Time Point | Result | Baseline | Result | Baseline | Result | Baseline |
| | Baseline^{[1]} | | | | | | |
| | n | 46 | | 49 | | 42 | |
| | Mean (SD) | 0.974 | | 1.016 | | 1.031 | |
| | | (0.174) | | (0.188) | | (0.174) | |
| | SEM | 0.026 | | 0.027 | | 0.027 | |
| | Median | 0.943 | | 0.993 | | 1.012 | |
| | Min, Max | 0.599, 1.416 | | 0.701, 1.938 | | 0.731, 1.474 | |
| | DB Month 12 | | | | | | |
| | n | 42 | 40 | 41 | 41 | 36 | 36 |
| | Mean (SD) | 0.971 | −0.009 | 1.013 | −0.014 | 1.003 | −0.012 |
| | | (0.181) | (0.040) | (0.201) | (0.041) | (0.181) | (0.032) |
| | SEM | 0.028 | 0.006 | 0.031 | 0.006 | 0.030 | 0.005 |
| | Median | 0.947 | −0.008 | 0.994 | −0.007 | 0.976 | −0.013 |
| | Min, Max | 0.585, 1.365 | −0.090, 0.163 | 0.714, 1.950 | −0.198, 0.034 | 0.726, 1.420 | −0.066, 0.062 |
| | LS Mean | | −0.01 | | −0.01 | | −0.01 |
| | (StdErr) | | (0.01) | | (0.01) | | (0.01) |
| | 95% CI | | (−0.02, 0.01) | | (−0.03, 0.01) | | (−0.03, 0.01) |
| | OCA - | | | | −0.01 | | 0.00 |
| | Placebo | | | | (0.01) | | (0.01) |
| | (StdErr) | | | | | | |
| | 95% CI of | | | | (−0.02, 0.01) | | (−0.02, 0.01) |
| | Mean | | | | | | |
| | Difference | | | | | | |
| | p-value^{[2]} | | | | 0.5054 | | 0.6451 |

^{[1]}Baseline is defined as the Day 0 value prior to treatment.
^{[2]}P-value for comparing active treatments to placebo is obtained using an ANCOVA model with baseline value as a covariate and fixed effects for treatment and randomization strata factor.

Summary of Results

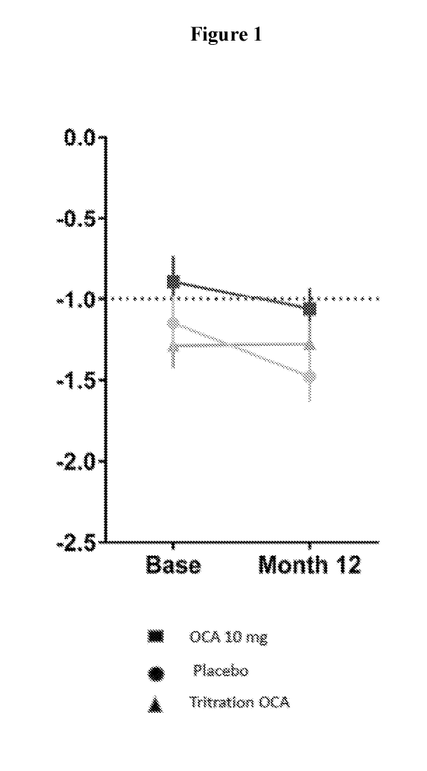

Given that osteoporosis occurs frequently in patients with PBC (20% to 30%) and the fracture incidence increases with advanced liver disease, bone density using Dual-emission x-ray absorptiometry (DEXA) scans were evaluated as an additional safety measure to evaluate if there was any worsening of osteoporosis or bone density in each individual patient. As DEXA scans were to be conducted at sites with the capabilities to perform this assessment, approximately 55% of subjects from the ITT population had scans performed at Baseline and Month 12. FIG. 1A provides DEXA demonstrated a smaller decrease in femoral bone mineral density T-score in both OCA groups versus placebo (p<0.05). Lumbar bone mineral density change was not significant between placebo and OCA groups. This preliminary analysis of bone mineral density in subjects treated with OCA suggests that OCA may attenuate the deterioration in femoral T-scores in subjects with PBC and merits further study.

1. A method of treating, reducing the risk of, preventing, or alleviating a symptom of a disease or condition associated with changes in bone density, osteoporosis, or an osteopenic disease in a subject in need of treatment thereof, comprising administering to the subject a therapeutically effective amount of a compound of formula I: 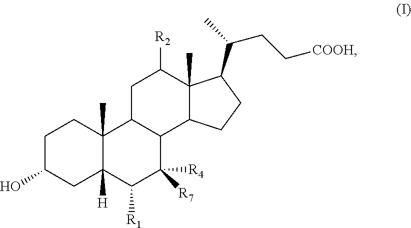
or a pharmaceutically acceptable salt or amino acid conjugate thereof, wherein:
R₁ is hydrogen or unsubstituted C₁-C₆ alkyl;
R₂ is hydrogen or α-hydroxyl;
R₄ is hydroxyl or hydrogen; and
R₇ is hydroxyl or hydrogen.

2. A method of inducing osteogenesis or bone growth in a subject, comprising administering to the subject in need of treatment thereof a therapeutically effective amount of a compound of formula I: 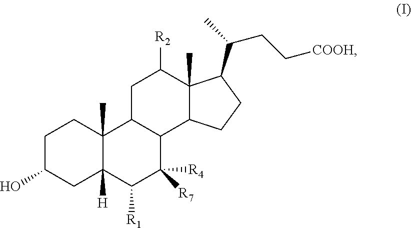
or a pharmaceutically acceptable salt, or amino acid conjugate thereof, wherein:
R₁ is hydrogen or unsubstituted C₁-C₆ alkyl;
R₂ is hydrogen or α-hydroxyl;
R₄ is hydroxyl or hydrogen; and
R₇ is hydroxyl or hydrogen.

3. A method of slowing, preventing, or reversing the reduction in bone density in a subject, comprising administering to the subject in need of treatment thereof a therapeutically effective amount of a compound of formula I: 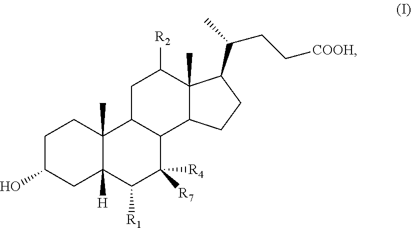
or a pharmaceutically acceptable salt or amino acid conjugate thereof, wherein:
R₁ is hydrogen or unsubstituted C₁-C₆ alkyl;
R₂ is hydrogen or α-hydroxyl;
R₄ is hydroxyl or hydrogen; and
R₇ is hydroxyl or hydrogen.

11. The method of claim 1, wherein the compound is 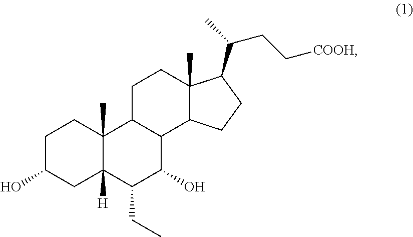 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]
or a pharmaceutically acceptable salt or amino acid conjugate thereof.

12. The method of claim 1, wherein the subject suffers from an FXR mediated disease or condition.

13. The method of claim 12, wherein the FXR mediated disease or condition is a liver disease.

14. The method of claim 13, wherein the liver disease is a cholestatic liver disease.

15. The method of claim 14, wherein the cholestatic liver disease is primary biliary cirrhosis or primary sclerosing cholangitis.

16. The method of claim 13, wherein the liver disease is a non-cholestatic liver disease.

17. The method of claim 1, wherein the compound of formula I, or a pharmaceutically acceptable salt or amino acid conjugate thereof, is administered in a total daily amount of 1-25 mg.

18. The method of claim 17, wherein the compound of formula I, or a pharmaceutically acceptable salt or amino acid conjugate thereof, is administered in an amount of about 5 mg or about 10 mg.

19. The method of claim 1, wherein the compound of formula I, or a pharmaceutically acceptable salt or amino acid conjugate thereof, is administered at a first dose for a first-time period, and at a second dose for a second time period.

20. The method of claim 19, wherein the first dose is a total daily amount of 1-25 mg.

21. The method of claim 20, wherein the first dose is about 5 mg.

22. The method of claim 19, wherein the second dose is a total daily amount of 1-25 mg.

23. The method of claim 22, wherein the second dose is about 10 mg.

24. The method of claim 19, wherein the first time period is from 1 month to 24 months.

25. The method of claim 24, wherein the first time period is about 6 months.

26. The method of claim 19, wherein the second time period is from 1 month to 24 months.

27. The method of claim 26, wherein the second time period is about 6 months.
